(19) United States
(12) Patent Application Publication
(10) Pub. No.: US 2023/0192623 A1
(43) Pub. Date: Jun. 22, 2023

(54) Indazole Derivatives

(71) Applicant: Hoffmann-La Roche Inc., Little Falls, NJ (US)

(72) Inventors: Stefan BERCHTOLD, Binningen (CH); Guido GALLEY, Rheinfelden (DE); Katrin GROEBKE ZBINDEN, Liestal (CH); Wolfgang GUBA, Muellheim (DE); Daniel HUNZIKER, Moehlin (CH); Daniela KRUMMENACHER, Zurich (CH)

(73) Assignee: Hoffmann-La Roche Inc., Little Falls, NJ (US)

(21) Appl. No.: 17/996,324

(22) PCT Filed: Apr. 14, 2021
(86) PCT No.: PCT/EP2021/059616

§ 371 Date: Oct. 14, 2022

(30) Foreign Application Priority Data
Apr. 16, 2020 (EP) 20169764.6

(51) Int. Cl. C07D 231/56; C07D 413/06; C07D 401/06

(52) U.S. Cl. CPC C07D 231/56; C07D 413/06; C07D 401/06

(57) ABSTRACT

The invention relates to a compound of formula (I) wherein R¹-R³ are as defined in the description and in the claims The compound of formula (I) can be used as a medicament.

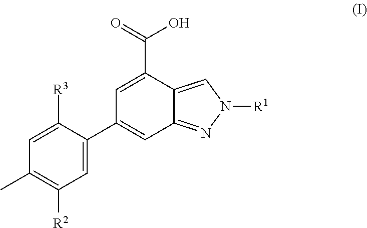

The present invention relates to organic compounds useful for therapy and/or prophylaxis in a mammal, and in particular to compounds that modulate cGAS activity.

The invention relates in particular to a compound of formula (I)

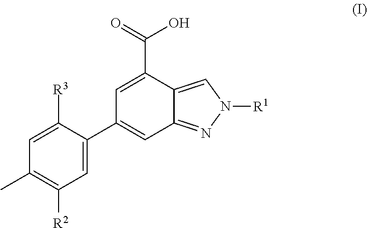

wherein
R¹ is hydrogen, alkylpiperazinylalkyl, halophenylalkyl, cycloalkyl(oxadiazolyl)alkyl, pyridinylalkyl, (dialkyloxazolyl)alkyl, haloalkyl or phenylalkyl;
R² is hydrogen or halogen; and
R³ is halogen; or a pharmaceutically acceptable salt, tautomer or ester thereof.

Cytokines are responsible for modulation of the innate immune response and the dysregulation of pro-inflammatory cytokines has been associated with severe systemic inflammation and autoimmune diseases, many of which lack efficient therapy as of today.

Vertebrates possess an innate and adaptive immune system as protection against pathogens and other challenges. The innate immune system is an evolutionary old system that is present beyond vertebrates. Unlike the adaptive immune system, it does not require priming or training, but works as a general physical barrier (e.g. skin) or by detection of specific patterns. One universal pattern to trigger the innate immune system is the detection of cytosolic double stranded DNA, which leads to Type I Interferon response. Sources of cytosolic dsDNA could be from bacterial or viral infection but as well accumulated self-DNA.

The cytosolic enzyme cyclic GMP-AMP Synthase (cGAS) is a sensor for cytosolic double stranded DNA. Binding of dsDNA results in the generation of the cyclic di-nucleotide 2,3-cGAMP by enzymatic linkage of ATP and GTP. 2,3-cGAMP acts as secondary messenger and binds to the Stimulator of Interferon Genes (STING), which resides in the endoplasmatic reticulum. Upon binding of 2,3-cGAMP, STING translocates to the perinuclear Golgi, where it associates with the TANK binding kinase 1 (TBK1) and recruits and phosphorylates Interferon Response Factor 3 (IRF3). Ultimately this results in the production of Type I Interferon (I IFN), other cytokines like IL-6, TNFα, IL1β and chemokines—essential factors for host defense against invading pathogens. However, inappropriate or chronic production of type I IFN and other pro-inflammatory cytokines are associated with severe systemic inflammation and autoimmune diseases. For instance, IFN signaling is involved in SLE, cutaneous skin diseases (dermatomyositis, and cutaneous lupus), interstitial pulmonary fibrosis, Sjogren syndrome, and type I diabetes (G. Trinchieri, J Exp Med. 2010 207(10): 2053-63). Other pro-inflammatory cytokine such as TNFα and IL1β play an important role in inflammatory bowel disease, NASH, juvenile inflammatory arthritis, ankylosing spondylitis and gout.

Chronic activation of cGAS/STING causes severe systemic inflammation. Evidence for its role in inflammation in the clinic comes from monogenic diseases. Patients with deficiencies in nucleic acid modifying enzymes, like Trexl, RNaseH2 and SAMHD1, suffer from Aicardi-Goutieres syndrome (AGS). The involvement of cGAS/STING was supported in Trex1 deficient mice that serve as a model for AGS.

Inhibition of the cGAS pathway which is upstream from the disease mediating cytokines is therefore a novel strategy in treating patients from multiple autoimmune diseases. Indications could include those linked to IFN signaling or those driven by TNFa and IL1β.

As of today many diseases caused by dysregulation of the innate immune system lack efficient therapies.

The compound of the invention binds to and modulates cGAS activity.

The compound of formula (I) is particularly useful in the treatment or prophylaxis of e.g. systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert.-butyl, sec.-butyl, the isomeric pentyls, the isomeric hexyls, the isomeric heptyls and the isomeric octyls, particularly methyl, ethyl, propyl, butyl and pentyl. Particular examples of alkyl are methyl, ethyl and propyl. Methyl and ethyl are particular examples of “alkyl” in the compound of formula (I).

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl and cyclohexyl, cycloheptyl and cyclooctyl. A particular example of “cycloalkyl” is cyclopropyl.

The term “oxy”, alone or in combination, signifies the —O— group.

The terms “halogen” or “halo”, alone or in combination, signifies fluorine, chlorine, bromine or iodine and particularly fluorine, chlorine or bromine, more particularly fluorine or chlorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. A particular “haloalkyl” is fluoroethyl.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid, acetic acid, sodium and potassium.

The term “pharmaceutically acceptable esters” means that compounds of general formula (I) may be derivatised at functional groups to provide derivatives which are capable of conversion back to the parent compounds in vivo. Examples of such compounds include physiologically acceptable and metabolically labile ester derivatives, such as methoxymethyl esters, methylthiomethyl esters and pivaloyloxymethyl esters. Additionally, any physiologically acceptable equivalents of the compounds of general formula (I), similar to the metabolically labile esters, which are capable of producing the parent compounds of general formula (I) in vivo, are within the scope of this invention.

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, N.Y.) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz). A particularly preferred protection group is tert-butoxycarbonyl (Boc).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The compound of formula (I) can exist as a tautomer (I′), i.e. a structural isomer which interconverts with the the compound of formula (I), in particular in solution.

The tautomeric equilibrium of the compound of formula (I) with its tautomeric form (I′) can be represented as follows:

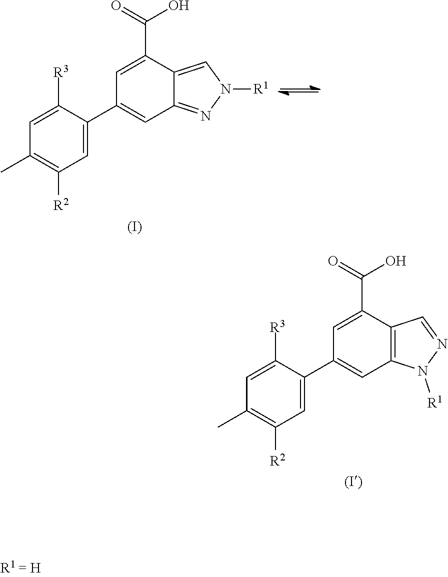

The term “asymmetric carbon atom” means a carbon atom with four different substituents. According to the Cahn-Ingold-Prelog Convention an asymmetric carbon atom can be of the “R” or “S” configuration.

The invention thus relates to:

A compound according to the invention wherein R¹ is hydrogen, methylpiperazinylethyl, chlorophenylmethyl, cyclopropyl(oxadiazolyl)methyl, pyridinylmethyl, (dimethyloxazolyl)methyl, trifluoroethyl, phenylmethyl or phenylethyl;

A compound according to the invention wherein R¹ is phenylalkyl;

A compound according to the invention wherein R¹ is phenylmethyl or phenylethyl;

A compound according to the invention wherein R² is hydrogen or fluorine;

A compound according to the invention wherein R² is hydrogen; and

A compound according to the invention wherein R³ is chlorine;

A compound according to the invention selected from
6-(2-chloro-4-methylphenyl)-2-[2-(4-methylpiperazin-1-yl)ethyl]indazole-4-carboxylic acid;
6-(2-chloro-4-methylphenyl)-2-[(2-chlorophenyl)methyl]indazole-4-carboxylic acid;
6-(2-chloro-4-methylphenyl)-2-[(3-chlorophenyl)methyl]indazole-4-carboxylic acid;
6-(2-chloro-4-methylphenyl)-2-[(4-chlorophenyl)methyl]indazole-4-carboxylic acid;
6-(2-chloro-4-methylphenyl)-2-[(3-cyclopropyl-1,2,4-oxadiazol-5-yl)methyl]indazole-4-carboxylic acid;
6-(2-chloro-4-methylphenyl)-2-(pyridin-3-ylmethyl)indazole-4-carboxylic acid;
6-(2-chloro-4-methylphenyl)-2-(pyridin-2-ylmethyl)indazole-4-carboxylic acid;
6-(2-chloro-4-methylphenyl)-2-[(4,5-dimethyl-1,3-oxazol-2-yl)methyl]indazole-4-carboxylic acid;
6-(2-chloro-4-methylphenyl)-2-(2,2,2-trifluoroethyl)-2H-indazole-4-carboxylic acid;
2-benzyl-6-(2-chloro-4-methylphenyl)-2H-indazole-4-carboxylic acid;
6-(2-chloro-4-methylphenyl)-2-phenethyl-2H-indazole-4-carboxylic acid;
6-(2-chloro-5-fluoro-4-methylphenyl)-1H-indazole-4-carboxylic acid; and
6-(2-chloro-4-methylphenyl)-1H-indazole-4-carboxylic acid;

or a pharmaceutically acceptable salt, tautomer or ester thereof.

A compound according to the selection from
2-benzyl-6-(2-chloro-4-methylphenyl)-2H-indazole-4-carboxylic acid; and
6-(2-chloro-4-methylphenyl)-2-phenethyl-2H-indazole-4-carboxylic acid;

or a pharmaceutically acceptable salt, tautomer or ester thereof.

The synthesis of the compound of formula (I) can, for example, be accomplished according to the following schemes.

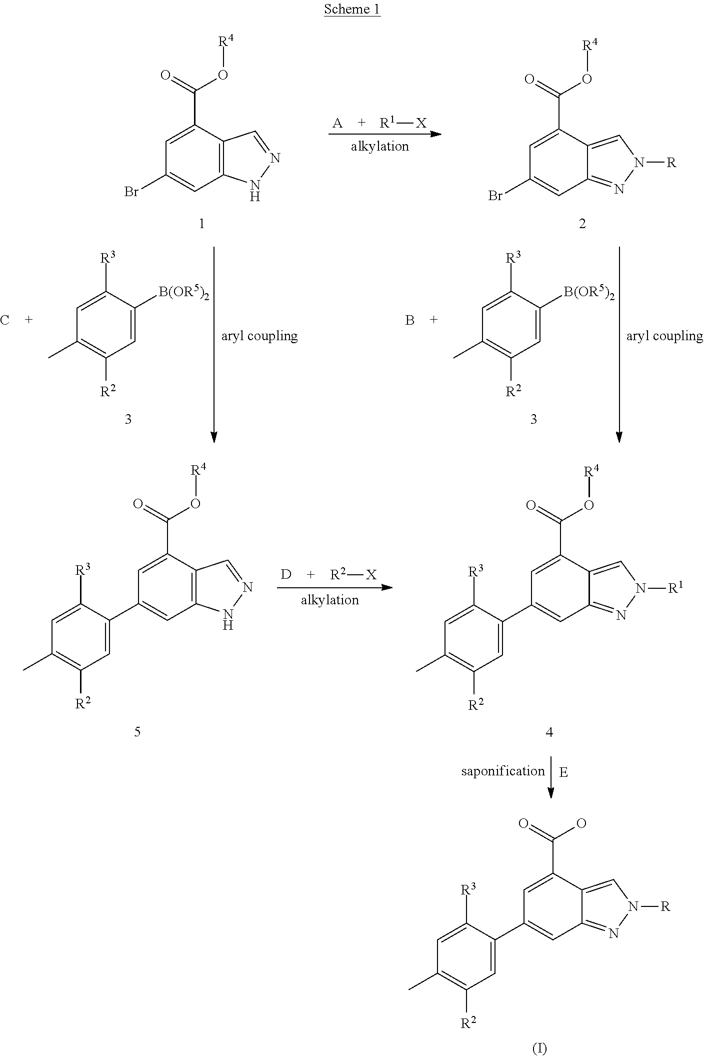

In scheme 1, R¹, R² and R³ are as defined above, wherein R¹ is not hydrogen; R⁴ is alkyl; R⁵ is hydrogen or alkyl.

In scheme 1, R⁴ is conveniently methyl.

In scheme 1, R⁵ is conveniently hydrogen.

Step A: The alkylation/benzylation can be accomplished by reacting the indazole 1 with a suitable substituted alkyl or substituted benyzl R¹—X such as substituted alkyl chlorides, alkyl bromides, substituted alkyl iodides, substituted alkyl tosylates, substituted benzyl bromides, substituted benzyl chlorides or the like and a base such as cesium carbonate, potassium carbonate, sodium carbonate, triethylamine or ethyldiisopropylamine in a solvent such as as dioxane, dimethylacetamide, dimethylformamide, tetrahydrofuran at 0° C.-150° C. for 1 h to 18 h. If regioisomeric mixtures of alkylation products are obtained they can be separated by column chromatography on silica gel using mixtures of organic solvents such as heptane, ethylacetate, methanol and dichloromethane to yield the described regioisomer as a pure compound.

Preferred conditions are the use of substituted alkyl chlorides or benzyl chlorides and cesium carbonate in dimethylformamide at 120° C. for several hours followed by chromatographic separation from the regioisomer.

Step B: Coupling of the bromoderivative 1 with a suitable boronic acid or boronic acid ester 3 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Preferred conditions are the use of tris(dibenzylideneacetone)dipalladium-chloroform adduct, X-phos and potassium phosphate in a mixture of dioxane and water at 110° C. for 1.5 h.

Step C: Coupling of the bromoderivative 2 with a suitable boronic acid or boronic acid ester 3 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Preferred conditions are the use of tris(dibenzylideneacetone)dipalladium-chloroform adduct, X-phos and potassium phosphate in a mixture of dioxane and water at 110° C. for 1.5 h.

Step D: The alkylation/benzylation can be accomplished by reacting the indazole 5 with a suitable substituted alkyl or substituted benzyl R¹—X such as substituted alkyl chlorides, alkyl bromides, substituted alkyl iodides, substituted alkyl tosylates, substituted benzyl bromides, substituted benzyl chlorides or the like and a base such as cesium carbonate, potassium carbonate, sodium carbonate, triethylamine or ethyldiisopropylamine in a solvent such as as dioxane, dimethylacetamide, dimethylformamide, tetrahydrofuran at 0° C.-150° C. for 1 h to 18 h. If regioisomeric mixtures of alkylation products are obtained they can be separated by column chromatography on silica gel using mixtures of organic solvents such as heptane, ethylacetate, methanol and dichloromethane to yield the described regioisomer as a pure compound.

Preferred conditions are the use of substituted alkyl chlorides or benzyl chlorides and cesium carbonate in dimethylformamide at 120° C. for several hours followed by chromatographic separation from the regioisomer.

Step E: Saponification can be accomplished by reaction of the alkyl ester 4 with a base such as lithium hydroxide, sodium hydroxide, potassium hydroxide or the like in a suitable solvent such as water, tetrahydrofuran, ethanol, methanol or mixtures thereof for 1-18 h at 0° C. to 70° C. Saponification can be furthermore accomplished by reacting the alkyl ester 4 with an acid such as hydrobromic acid or hydrochloric acid in water or acetic acid or a mixture thereof at 20° C.-110° C. for 1-24 h. Preferred conditions are the use of lithium hydroxide in a mixture of tetrahydrofuran and water at 65° C. for 4 h.

The compound of formula (I-1), which is a tautomer of the compound (I) wherein le is hydrogen, can be synthesised according to the following scheme:

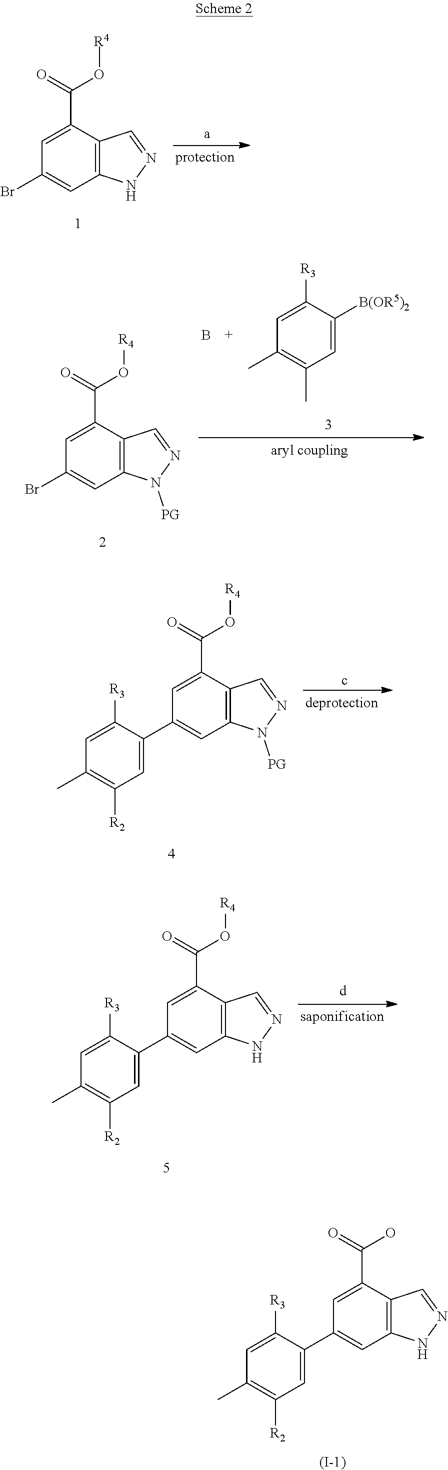

In scheme 2, R¹ is hydrogen, R² and R³ are as defined abov; R⁴ is alkyl; R⁵ is hydrogen or alkyl.

In scheme 2, R⁴ is conveniently methyl.

In scheme 2, R⁵ is conveniently hydrogen.

Step A: Protection of the nitrogen can be accomplished by reacting indazol 1 with a suitable reagent such as di-tert-butyl-dicarbonate in presence of a base such as cesium carbonate, 4-dimethylaminopyridine, triethylamine or ethyl diisopropylamine at 0° C.-75° C. in a suitable solvent such as dichloromethane, dichloroethane, tetrahydrofuran or acetonitrile for 1 h to 2 days.

A preferred protecting group is the tert.-butyloxycarbonyl group and preferred conditions for the introduction are the use of di-tert-butyl-dicarbonate and cesium carbonate in dioxane for 45 min at room temperature.

Step B: Coupling of the bromoderivative 2 with a suitable boronic acid or boronic acid ester 3 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Preferred conditions are the use of tris(dibenzylideneacetone)dipalladium-chloroform adduct, X-phos and potassium phosphate in a mixture of dioxane and water at 110° C. for 1.5 h.

Step C: Deprotection can be accomplished by reaction of benzimidazole 4 with a suitable reagent such as trifluoroacetic acid or hydrochloric acid in dichloromethane or dioxane at room temperature for 1-18 h in case the preferred tert.-butyloxycarbonyl group (PG=Boc) has been used.

Preferred conditions are the heating of the compound use of hydrochloric acid in dioxane at 20° C. for 18 h.

Step D: Saponification can be accomplished by reaction of the alkyl ester 5 with a base such as lithium hydroxide, sodium hydroxide, potassium hydroxide or the like in a suitable solvent such as water, tetrahydrofuran, ethanol, methanol or mixtures thereof for 1-18 h at 0° C. to 70° C. Saponification can be furthermore accomplished by reacting the alkyl ester 5 with an acid such as hydrobromic acid or hydrochloric acid in water or acetic acid or a mixture thereof at 20° C.-110° C. for 1-24 h.

Preferred conditions are the use of hydrobromic acid in acetic acid at 110° C. for 18 h.

The invention thus also relates to a process for the preparation of a compound according to the invention, comprising the saponification of a compound of formula (Al) or its tautomer.

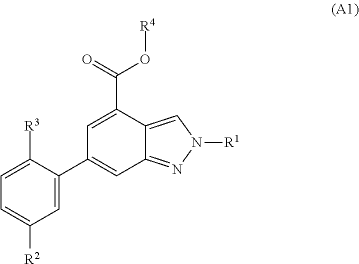

in a suitable solvent in the presence of a base or a acid;

wherein R¹, R² and R³ are as defined above and R⁴ is alkyl.

R⁴ is conveniently methyl.

The solvent of the saponification is conveniently water, tetrahydrofuran, ethanol, methanol, acetic acid or mixtures thereof.

In the saponification the base can be for example lithium hydroxide, sodium hydroxide or potassium hydroxide.

In the saponification the acid can be for example hydrobromic acid or hydrochloric acid.

Convenient conditions for the saponification are 0° C.-110° C. for 1-24 h.

Preferred conditions for saponification under basic conditions are the use of lithium hydroxide in a mixture of tetrahydrofuran and water at 65° C. for 4 h.

Preferred conditions for saponification under acid conditions are the use of hydrobromic acid in acetic acid at 110° C. for 18 h.

The invention also relates to a compound according to the invention when manufactured according to a process of the invention.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The invention also relates in particular to:

A compound of formula (I) for use in the treatment of a disease modulated by cGAS;

The use of a compound of formula (I) for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

The use of a compound of formula (I) for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

A compound of formula (I) for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS); and

A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS); which method comprises administering an effective amount of a compound of formula (I) to a patient in need thereof.

The invention will now be illustrated by the following examples which have no limiting character.

### EXAMPLES

Abbreviations

DMSO=dimethyl sulfoxide; ESI=electrospray ionization; EtOAc=ethyl acetate; MS=mass spectrometry; RT=room temperature; THF=tetrahydrofuran.

### Example 1

### 6-(2-Chloro-4-methylphenyl)-2-(2-(4-methylpiperazin-1-yl)ethyl)indazole-4-carboxylic acid

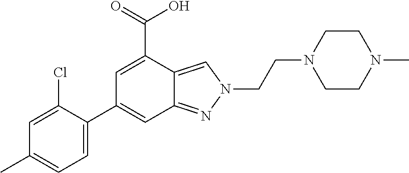

### a) Methyl 6-(2-chloro-4-methylphenyl)-1H-indazole-4-carboxylate

In a 100 ml screw glass vial, methyl 6-bromo-1H-indazole-4-carboxylate (2 g, 7.84 mmol, Eq: 1), (2-chloro-4-methylphenyl)boronic acid (1.34 g, 7.84 mmol, Eq: 1) and potassium phosphate (tribasic) (3.33 g, 15.7 mmol, Eq: 2) were combined with dioxane (32 ml) and water (8 ml) . The vial was degassed with argon before X-phos (187 mg, 392 μmol, Eq: 0.05) and tris(dibenzylideneacetone)dipalladium-chloroform (203 mg, 196 μmol, Eq: 0.025) were added. The vial was closed and the reaction mixture was heated to 110 ° C. and stirred for 1.5 h. The reaction mixture was poured into 80 ml of water and extracted with EtOAc (3×80 ml). The organic layers were combined, dried over MgSO₄, filtered through sintered glass, concentrated and dried in vacuo. The crude material was purified by flash chromatography (silica gel, 80 g, 0% to 60% EtOAc in heptane). The fractions were combined, concentrated and dried in vacuo to afford the title compound methyl 6-(2-chloro-4-methylphenyl)-1H-indazole-4-carboxylate (2.01 g, 6.32 mmol, 80.6% yield) as off-white solid, MS (ESI): 301.074 [M+H]+.

### b) Methyl 6-(2-chloro-4-methylphenyl)-2-[2-(4-methylpiperazin-1-yl)ethyl]indazole carboxylate

Methyl 6-(2-chloro-4-methylphenyl)-1H-indazole-4-carboxylate (150 mg, 499 Eq: 1) and cesium carbonate (488 mg, 1.5 mmol, Eq: 3) were combined with dimethylformamide (2.5 ml) to give a brown suspension. Then 1-(2-chloroethyl) methylpiperazine (81.1 mg, 499 μmol, Eq: 1) was added at room temperature. The reaction mixture was stirred overnight at room temperature and to 120° C. for 4.5 hours. The mixture was poured into 30 ml of water and extracted with EtOAc (two times 30 ml). The organic layer was back-extracted with saturated NaC1 solution (30 ml) and dried over Na₂SO₄ and concentrated in vacuo. The crude material was purified by flash chromatography (silica gel, 12 g, 0% to 10% MeOH in dichloromethane) to afford the title compound ethyl 6-(2-chloro-4-methylphenyl)-2-[2-(4-methylpiperazin-1-yl)ethyl]indazole-4-carboxylate (14.2 mg, 33 μmol, 6.6% yield) as brown oil, MS (ESI): 427.1889 [M+H]+.

### c) 6-(2-Chloro-4-methylphenyl)-2-(2-(4-methylpiperazin-1-yl)ethyl)indazole-4-carboxylic acid

To a solution of methyl 6-(2-chloro-4-methylphenyl)-2-(2-(4-methylpiperazin-1-yl)ethyl)-2H-indazole-4-carboxylate (12 mg, 28.1 μmol, Eq: 1) in tetrahydrofuran (2 ml) was added lithium hydroxide monohydrate (2.36 mg, 56.2 Eq: 2) dissolved in water (0.5 ml). The reaction mixture was heated to 65° C. and stirred during 4 hours. The mixture was quenched with HCl (2M, 28.1 56.2 μmol, Eq: 2) and concentrated in vacuo. The crude material was triturated with diethyl ether (2×10 ml) to obtain the title compound 6-(2-chloro-4-methylphenyl)-2-(2-(4-methylpiperazin-1-yl)ethyl)indazole-4-carboxylic acid (11.4 mg, 25.5 μmol, 90.7% yield) as off-white solid, MS (ESI): 413.23 [M+H]+.

### Example 2

### 6-(2-Chloro-4-methylphenyl)-2-[(2-chlorophenyl)methyl]indazole-4-carboxylic acid

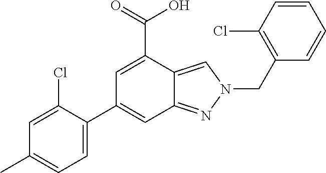 [SMILES: Cc1ccc(-c2cc(C(=O)O)c3cn(Cc4ccccc4Cl)nc3c2)c(Cl)c1]

The title compound was obtained in comparable yield analogous to the procedure described for Example 1 using 2-chlorobenzylbromide instead of 1-(2-chloroethyl) methylpiperazine in step b), white solid, (MS (ESI): 411.066 [M+H]+.

### Example 3

### 6-(2-Chloro-4-methylphenyl)-2-[(3-chlorophenyl)methyl]indazole-4-carboxylic acid

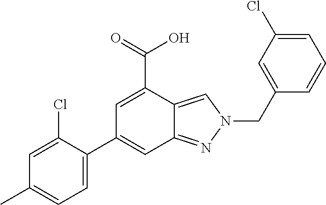 [SMILES: Cc1ccc(-c2cc(C(=O)O)c3cn(Cc4cccc(Cl)c4)nc3c2)c(Cl)c1]

The title compound was obtained in comparable yield analogous to the procedure described for Example 1 using 3-chlorobenzylbromide instead of 1-(2-chloroethyl)-4-methylpiperazine in step b), white solid, (MS (ESI): 411.066 [M+H]+.

### Example 4

### 6-(2-Chloro-4-methylphenyl)-2-[(4-chlorophenyl)methyl]indazole-4-carboxylic acid

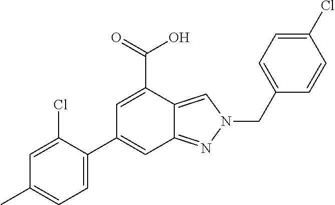 [SMILES: Cc1ccc(-c2cc(C(=O)O)c3cn(Cc4ccc(Cl)cc4)nc3c2)c(Cl)c1]

The title compound was obtained in comparable yield analogous to the procedure described for Example 1 using 4-chlorobenzylbromide instead of 1-(2-chloroethyl) methylpiperazine in step b), white solid, (MS (ESI): 411.066 [M+H]+.

### Example 5

### 6-(2-Chloro-4-methylphenyl)-2-((3-cyclopropyl-1,2,4-oxadiazol-5-yl)methyl)indazole-4-carboxylic acid

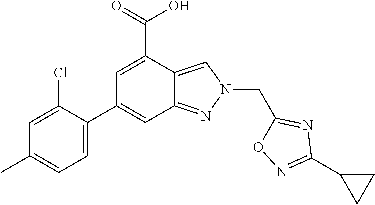 [SMILES: Cc1ccc(-c2cc(C(=O)O)c3cn(Cc4nc(C5CC5)no4)nc3c2)c(Cl)c1]

The title compound was obtained in comparable yield analogous to the procedure described for Example 1 using 5-(chloromethyl)-3-cyclopropyl-1,2,4-oxadiazole instead of 1-(2-chloroethyl)-4-methylpiperazine in step b), light yellow solid, (MS (ESI): 409.14 [M+H]+.

### Example 6

### 6-(2-Chloro-4-methylphenyl)-2-(pyridin-3-ylmethyl)indazole-4-carboxylic acid

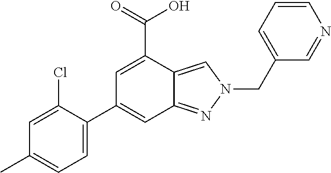 [SMILES: Cc1ccc(-c2cc(C(=O)O)c3cn(Cc4cccnc4)nc3c2)c(Cl)c1]

The title compound was obtained in comparable yield analogous to the procedure described for Example 1 using 3-(chloromethyl)pyridine hydrochloride instead of 1-(2-chloroethyl)-4-methylpiperazine in step b), white solid, (MS (ESI): 378.100 [M+H]+.

### Example 7

### 6-(2-Chloro-4-methylphenyl)-2-(pyridin-2-ylmethyl)indazole-4-carboxylic acid

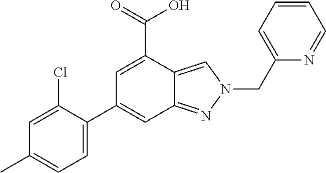 [SMILES: Cc1ccc(-c2cc(C(=O)O)c3cn(Cc4ccccn4)nc3c2)c(Cl)c1]

### a) Methyl 6-(2-chloro-4-methylphenyl)-2-(pyridin-2-ylmethyl)-2H-indazole-4-carboxylate

Methyl 6-(2-chloro-4-methylphenyl)-1H-indazole-4-carboxylate (see Example 1; 150 mg, 499 μmol, Eq: 1) and cesium carbonate (244 mg, 748 μmol, Eq: 1.5) were combined with dimethylformamide (2.5 ml) to give a brown suspension. Then 2-(bromomethyl)pyridine (129 mg, 748 μmol, Eq: 1.5) was added at room temperature. The reaction mixture was stirred overnight. Additional 2-(bromomethyl)pyridine (42.9 mg, 249 μmol, Eq: 0.5) and cesium carbonate (81.3 mg, 249 μmol, Eq: 0.5) were added and the RM was stirred at 60° C. for 3h. The reaction mixture was poured into 30 ml H₂O and extracted with EtOAc (2×30 ml). The organic layer was back-extracted with saturated NaCl solution (1×30 ml) and dried over Na₂SO₄ and concentrated in vacuo. The crude material was purified by flash chromatography (silica gel, 25 g, 0% to 70% EtOAc in Heptane) to afford the title compound methyl 6-(2-chloro-4-methylphenyl)-2-(pyridin-2-ylmethyl)-2H-indazole-4-carboxylate (74.6 mg, 195 μmol, 39.0% yield) as white solid, MS (ESI): 392.1169 [M+H]+.

### b) 6-(2-Chloro-4-methylphenyl)-2-(pyridin-2-ylmethyl)indazole-4-carboxylic acid

To a solution of methyl 6-(2-chloro-4-methylphenyl)-2-(pyridin-2-ylmethyl)-2H-indazole-4-carboxylate (37.8 mg, 96.5 μmol, Eq: 1) in tetrahydrofuran (2 ml) was added lithium hydroxide monohydrate (8.9 mg, 212 μmol, Eq: 2.2) solved in water (1 ml). The reaction mixture was warmed to 65° C. and stirred over night. HCl 2M (96.5 193 μmol, Eq: 2) was added and concentrated in vacuo. The reaction mixture was poured into 2 ml of water and extracted with methyltetrahydrofuran (4×5 ml). The organic layers were dried over Na2SO4 and concentrated in vacuo. Diethylether (3 ml) were added and the suspension was filtered through a Sartorious filter to get 11.5 mg of 6-(2-chloro-4-methylphenyl) (pyridin-2-ylmethyl)indazole-4-carboxylic acid (11.5 mg, 29.4 μmol, 30.5% yield) as white solid. MS (ESI): 378.10 [M+H]+.

### Example 8

### 6-(2-Chloro-4-methylphenyl)-2-((4,5-dimethyloxazol-2-yl)methyl)indazole-4-carboxylic acid

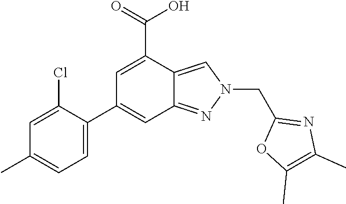

The title compound was obtained in comparable yield analogous to the procedure described for Example 1 using 2-(chloromethyl)-4,5-dimethyloxazole instead of 1-(2-chloroethyl)-4-methylpiperazine in step b), light yellow solid, (MS (ESI): 396.16 [M+H]+.

### Example 9

### 6-(2-Chloro-4-methylphenyl)-2-(2,2,2-trifluoroethyl)-2H-indazole-4-carboxylic acid

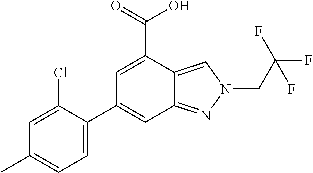 [SMILES: Cc1ccc(-c2cc(C(=O)O)c3cn(CC(F)(F)F)nc3c2)c(Cl)c1]

### a) Methyl 6-bromo-2-(2,2,2-trifluoroethyl)-2H-indazole-4-carboxylate

In a three-necked flask, methyl 6-bromo-1H-indazole-4-carboxylate (400 mg, 1.57 mmol, Eq: 1) and potassium carbonate (482 mg, 3.45 mmol, Eq: 2.2) were combined with dimethylformamide (10 ml) to give a orange suspension. Then 2,2,2-trifluoroethyl trifluoromethanesulfonate (413 mg, 243 1.73 mmol, Eq: 1.1) was added at room temperature. The mixture was stirred at r.t. for 4.5 hours. Then the RM was concentrated in vacuo. The residue was taken up in EtOAc and water, the aqueous layer was extracted again with EtOAc, the combined org. layers were washed with water and brine, dried over Na₂SO₄, filtrated and evaporated. The residue was diluted with dichloromethane, evaporated with silica gel to dryness and transferred to a column. Flash chromatography (silica gel, 40 g, 0% to 30% EtOAc in Heptane) yielded title compound methyl 6-bromo-2-(2,2,2-trifluoroethyl)-2H-indazole-4-carboxylate (eluating after the other regioisomer, 210 mg, 623 μmol, 39.7% yield) as white solid. MS (ESI): 337.0, 339.0 [M+H]+.

### b) Methyl 6-(2-chloro-4-methylphenyl)-2-(2,2,2-trifluoroethyl)-2H-indazole-4-carboxylate

Methyl 6-bromo-2-(2,2,2-trifluoroethyl)-2H-indazole-4-carboxylate (80 mg, 237 μmol, Eq: 1) and (2-chloro-4-methylphenyl)boronic acid (60.7 mg, 356 μmol, Eq: 1.5) were solved in 1,4-dioxane (6 ml) and water (3 ml). Cesium carbonate (312 mg, 949 μmol, Eq: 4) was added and the mixture was degassed by bubbling argon through the mixture (5 min), then 1,1′-bis(diphenylphosphino)ferrocene-palladium(II)dichloride dichloromethane complex (9.69 mg, 11.9 μmol, Eq: 0.05) was added. The reaction was stirred in a sealed tube at 90° C. for 15 min. The reaction mixture was taken up in EtOAc and washed with sat. NH₄Cl solution and brine, the organic layer was dried over Na₂SO₄, filtrated and evaporated. The residue was purified by flash chromatography (silica gel, 40 g, 0% to 30% EtOAc in Heptane) to afford the title compound methyl 6-(2-chloro-4-methylphenyl)-2-(2,2,2-trifluoroethyl)-2H-indazole-4-carboxylate (78.5 mg, 191 μmol, 80.5% yield) as white solid. MS (ESI): 383.1 [M+H]+.

### c) 6-(2-Chloro-4-methylphenyl)-2-(2,2,2-trifluoroethyl)-2H-indazole-4-carboxylic acid

Methyl 6-(2-chloro-4-methylphenyl)-2-(2,2,2-trifluoroethyl)-2H-indazole-4-carboxylate (74.2 mg, 194 μmol, Eq: 1) was solved in THF (3.3 ml). At room temperature lithium hydroxide (1M, 291 μmol, 291 μmol, Eq: 1.5) was added. The reaction mixture was stirred at 65° C. For work-up the mixture was diluted with water and extracted two times with diethylether, the organic layer was washed with water and the combined aqueous layers were acidified with HCl (2M, 145 μl, 291 μmol, Eq: 1.5) and the pH was adjusted to 3, extracted with EtOAc, the combined organic layers were dried over Na₂SO₄, filtrated and evaporated to afford the title compound 6-(2-chloro-4-methylphenyl)-2-(2,2,2-trifluoroethyl)-2H-indazole-4-carboxylic acid (70.4 mg, 205 μmol, 86.7% yield) as white solid. MS (ESI): 369.1 [M+H]+.

### Example 10

### 2-Benzyl-6-(2-chloro-4-methylphenyl)-2H--indazole-4-carboxylic acid

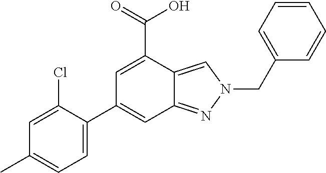 [SMILES: Cc1ccc(-c2cc(C(=O)O)c3cn(Cc4ccccc4)nc3c2)c(Cl)c1]

The title compound was obtained in comparable yield analogous to the procedure described for Example 9 using benzyl bromide instead of 2,2,2-trifluoroethyl trifluoromethanesulfonate and cesium carbonate instead of potassium carbonate in step a), white solid, MS (ESI): 377.2 [M+H]+.

### Example 11

### 6-(2-Chloro-4-methylphenyl)-2-phenethyl-2H-indazole-4-carboxylic acid

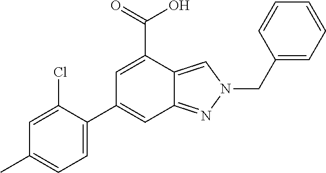 [SMILES: Cc1ccc(-c2cc(C(=O)O)c3cn(Cc4ccccc4)nc3c2)c(Cl)c1]

The title compound was obtained in comparable yield analogous to the procedure described for Example 9 using phenethyl bromide instead of 2,2,2-trifluoroethyl trifluoromethanesulfonate and acetonitrile instead of dioxane in step a), white solid, MS (ESI): 391.2 [M+H]+.

### Example 12

### 6-(2-Chloro-5-fluoro-4-methylphenyl)-1H-indazole-4-carboxylic acid

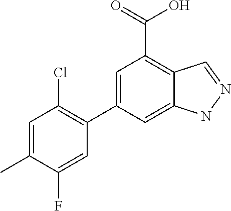 [SMILES: Cc1cc(Cl)c(-c2cc(C(=O)O)c3cn[nH]c3c2)cc1F]

### a) 1-(tert-Butyl) 4-methyl 6-(2-chloro-5-fluoro-4-methylphenyl)-1H-indazole-1,4-dicarboxylate

Methyl 6-bromo-1H-indazole-4-carboxylate (100 mg, 392 Eq: 1) was solved in 1,4-dioxane (3 ml). At room tempearature cesium carbonate (452 mg, 1.37 mmol, Eq: 3.5) and di-tert-butyl-dicarbonate (96 mg, 431 μmol, Eq: 1.1) were added. The reaction mixture was stirred 45min at room temperature, then (2-chloro-5-fluoro-4-methylphenyl)boronic acid (82.9 mg, 431 μmol, Eq: 1.1), Water (1 ml) and 1,1′-bis(diphenylphosphino)ferrocene-palladium(II)dichloride dichloromethane complex (32 mg, 39.2 μmol, Eq: 0.1) were added. The reaction was stirred in a sealed tube at 105°. For work-up the mixture was taken up in EtOAc and water, the aqueous layer was extracted two times with EtOAc, the combined organic layer was dried over Na₂SO₄, filtrated and evaporated. The residue was diluted with EtOAc and transferred to a column. Purification by flash chromatography (silica gel, 40 g, 0% to 80% EtOAc in Heptane) afforded the title compound 1-(tert-butyl) 4-methyl 6-(2-chloro-5-fluoro-4-methylphenyl)-1H-indazole-1,4-dicarboxylate (144.8 mg, 345 μmol, 88.2% yield) as white foam, MS (ESI): 363.1[M-^{t}Bu+H]+.

### b) Methyl 6-(2-chloro-5-fluoro-4-methylphenyl)-1H-indazole-4-carboxylate

1-(tert-Butyl) 4-methyl 6-(2-chloro-5-fluoro-4-methylphenyl)-1H-indazole-1,4-dicarboxylate (130.8 mg, 306 Eq: 1) was solved in 1,4-dioxane (1.4 ml). At room temperature HCl in dioxane (4M, 2.3 ml, 9.18 mmol, Eq: 30) was added and the mixture was stirred overnight at room temperature. For work-up the mixture was concentrated in vacuo. The residue was taken up in EtOAc, water and washed with saturated NaHCO₃-solution. The organic layer was dried over Na₂SO₄, filtrated and evaporated to afford the title compound methyl 6-(2-chloro-5-fluoro-4-methylphenyl)-1H-indazole-4-carboxylate (97.1 mg, 305 μmol, 99.5% yield) as white solid, MS (ESI): 319.1 [M+H]+.

### c) 6-(2-Chloro-5-fluoro-4-methylphenyl)-1H-indazole-4-carboxylic acid

Methyl 6-(2-chloro-5-fluoro-4-methylphenyl)-1H-indazole-4-carboxylate (35 mg, 110 μmol, Eq: 1) was solved in acetic acid (765 mg, 705 μl, 12.7 mmol, Eq: 116). Hydrobromic acid (529 mg, 353 μl, 3.14 mmol, Eq: 28.6) was added at room temperature. The reaction mixture was stirred at 110° C. overnight. The mixture was concentrated in vacuo. The residue was taken up in EtOAc and the organic layer was washed three times with water. The organic layer was dried over Na₂SO₄, filtrated, evaporated and dried on high vacuum to afford the title compound 6-(2-chloro-5-fluoro-4-methylphenyl)-1H-indazole-4-carboxylic acid (33.3 mg, 109 μmol, 99.1% yield) as white solid, MS (ESI): 305.1 [M+H]+.

### Example 13

### 6-(2-Chloro-4-methylphenyl)-1H-indazole-4-carboxylic acid

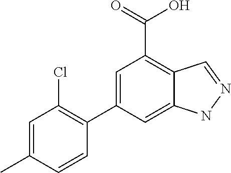 [SMILES: Cc1ccc(-c2cc(C(=O)O)c3cn[nH]c3c2)c(Cl)c1]

The title compound was obtained in comparable yield analogous to the procedure described for Example 12 using (2-chloro-4-methylphenyl)boronic acid instead of (2-chloro-5-fluoro-4-methylphenyl)boronic acid in step a), white solid, MS (ESI): 287.1 [M+H]+.

### Example 14

Malachite Green Assay to Measure cGAS Activity

Compounds were tested for cGAS inhibition in a coupled enzymatic assay based on Phosphate detection by Malachite Green. Final assay conditions were 20 mM TRIS pH 7.5 (Applichem), 5 mM MgCl₂ (Sigma) and 0.01% BSA (Sigma) supplemented with 80 μM ATP (Sigma), 80 μM GTP (Sigma) and 100 nM Interferon Stimulating DNA (ISD) (Microsynth). Recombinantly expressed purified human cGAS (residues 161-522) was used at 25nM.

All compounds were prepared as 10 mM stock solutions in DMSO and a 16 pt dilution series in DMSO with a dilution factor of 2.5 was prepared. 1 μL of DMSO dilution series was transferred to 32.3 μL reaction buffer, mixed by pipetting up/down, spun for 1 minute at 3000 rpm and was visually inspected for precipitation. 5 μL of 3-fold enzyme stock solution were transferred to an empty 384-well Black/Clear Flat Bottom Polystyrene NBS (Corning) rows 3-24. Rows 1-2 were filled with assay buffer. Plates were spun 10 seconds at 1000 rpm (164×g). 5 μL of compound intermediate dilution was added and mixed by pipetting up/down to rows 3-24. Rows 1-2 were filled with 3.1% DMSO assay buffer. Plates were spun 10 seconds at 1000 rpm (164×g). 5 μL 3-fold Nucleotide/DNA mix was added to all wells to start the reaction. Plates were spun 10 seconds at 1000 rpm (164×g) and incubated for 4 hour at room temperature (RT) in the dark. 5 μL 4U/mL PPase (Sigma) were added to all wells. Plates spun 10 seconds at 1000 rpm (164×g). 10 μL BioMol green Solution (Enzo Life Sciences) was added to all wells. Plates spun 10 seconds at 1000 rpm (164×g) and incubated 30 minutes at RT in the dark. Absorbance data was collected 620 nm on an EnVision Multilable Reader (Perkin Elmer) and the following measurement settings were used: excitation filter photometric was 620 nm; excitation from the top; measurement height was 1 mm; number of flashes was 30; number of flashes integrated was 1.

All plates are checked for abnormalities and outliers in the Blank Control (no protein, row 1) and the Neutral Control (no compound, row 2) are excluded using the 3*SD rule. Data was normalized to 0 and 100% by Blank and Neutral Control and each curve was fitted and judged using the 4 parameter logistic equation to determine the IC50 for cGAS inhibition.

The results of this assay are provided in Table 1. Table 1 provides IC50 values (μM) for cGAS inhibition obtained for particular examples of the present invention as measured by the above-described assay.

| Example | IC50 cGAS (μM) |
|---|---|
| 1 | 1.92 |
| 2 | 2.2 |
| 3 | 3.18 |
| 4 | 2.76 |
| 5 | 2.21 |
| 6 | 2.93 |
| 7 | 2.4 |
| 8 | 2.74 |
| 9 | 1.56 |
| 10 | 1.37 |
| 11 | 2.35 |
| 12 | 4.59 |
| 13 | 4.46 |

### Example A

Film coated tablets containing the following ingredients can be manufactured in a conventional manner:

| | |
|---|---|
| Ingredients | Per tablet |

Kernel:

| | | | | |
|---|---|---|---|---|
| Compound of formula (I) | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxide (yellow) | 0.8 | mg | 1.6 | mg |
| Titan dioxide | 0.8 | mg | 1.6 | mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution / suspension of the above mentioned film coat.

### Example B

Capsules containing the following ingredients can be manufactured in a conventional manner:

| Ingredients | Per capsule |
|---|---|
| Compound of formula (I) | 25.0 mg |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| | |
|---|---|
| Compound of formula (I) | 3.0 mg |
| Polyethylene glycol 400 | 150.0 mg |
| Acetic acid | q.s. ad pH 5.0 |
| Water for injection solutions | ad 1.0 ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

1. A compound of formula (I) 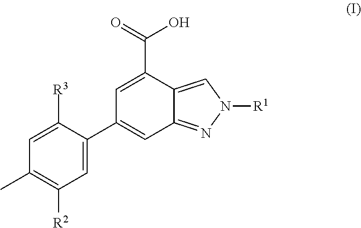
wherein
R¹ is hydrogen, alkylpiperazinylalkyl, halophenylalkyl, cycloalkyl(oxadiazolyl)alkyl, pyridinylalkyl, (dialkyloxazolyl)alkyl, haloalkyl or phenylalkyl;
R² is hydrogen or halogen; and
R³ is halogen;
or a pharmaceutically acceptable salt, tautomer or ester thereof

2. A compound according to claim 1, wherein R¹ is hydrogen, methylpiperazinylethyl, chlorophenylmethyl, cyclopropyl(oxadiazolyl)methyl, pyridinylmethyl, (dimethyloxazolyl)methyl, trifluoroethyl, phenylmethyl or phenyl ethyl.

3. A compound according to claim 1, wherein R¹ is phenylalkyl.

4. A compound according to any one of claims 1 to 3, wherein R¹ is phenylmethyl or phenylethyl.

5. A compound according to any one of claims 1 to 4, wherein R² is hydrogen.

6. A compound according to any one of claims 1 to 5, wherein R³ is chlorine.

7. A compound according to any one of claims 1 to 6 selected from
6-(2-chloro-4-methylphenyl)-2-[2-(4-methylpiperazin-1-yl)ethyl]indazole carboxylic acid;
6-(2-chloro-4-methylphenyl)-2-[(2-chlorophenyl)methyl]indazole-4-carboxylic acid;
6-(2-chloro-4-methylphenyl)-2-[(3-chlorophenyl)methyl]indazole-4-carboxylic acid;
6-(2-chloro-4-methylphenyl)-2-[(4-chlorophenyl)methyl]indazole-4-carboxylic acid;
6-(2-chloro-4-methylphenyl)-2-[(3-cyclopropyl-1,2,4-oxadiazol-5-yl)methyl]indazole-4-carboxylic acid;
6-(2-chloro-4-methylphenyl)-2-(pyridin-3-ylmethyl)indazole-4-carboxylic acid;
6-(2-chloro-4-methylphenyl)-2-(pyridin-2-ylmethyl)indazole-4-carboxylic acid;
6-(2-chloro-4-methylphenyl)-2-[(4,5-dimethyl-1,3-oxazol-2-yl)methyl]indazole-4-carboxylic acid;
6-(2-chloro-4-methylphenyl)-2-(2,2,2-trifluoroethyl)-2H-indazole-4-carboxylic acid;
2-benzyl-6-(2-chloro-4-methylphenyl)-2H-indazole-4-carboxylic acid;
6-(2-chloro-4-methylphenyl)-2-phenethyl-2H-indazole-4-carboxylic acid;
6-(2-chloro-5-fluoro-4-methylphenyl)-1H-indazole-4-carboxylic acid; and
6-(2-chloro-4-methylphenyl)-1H-indazole-4-carboxylic acid;
or a pharmaceutically acceptable salt, tautomer or ester thereof.

8. A compound according to any one of claims 1 to 8 selected from
2-benzyl-6-(2-chloro-4-methylphenyl)-2H-indazole-4-carboxylic acid; and
6-(2-chloro-4-methylphenyl)-2-phenethyl-2H-indazole-4-carboxylic acid;
or a pharmaceutically acceptable salt, tautomer or ester thereof.

9. A process for the preparation of a compound according to any one of claims 1 to 8, comprising the saponification of a compound of formula (A1) or its tautomer 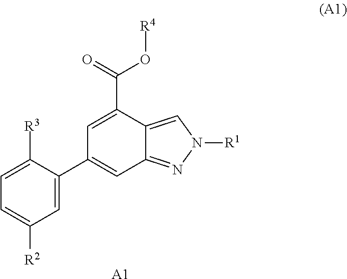
in a suitable solvent in the presence of a base or an acid;
wherein R¹, R² and R³ are as defined above and R⁴ is alkyl.

10. A compound according to any one of claims 1 to 8, when manufactured according to a process of claim 9.

11. A compound according to any one of claims 1 to 8 for use as therapeutically active sub stance.

12. A pharmaceutical composition comprising a compound in accordance with any one of claims 1 to 8 and a therapeutically inert carrier.

13. The use of a compound according to any one of claims 1 to 8 for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

14. The use of a compound according to any one of claims 1 to 8 for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

15. A compound according to any one of claims 1 to 8 for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

16. A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS), which method comprises administering an effective amout of a compound as defined in any one of claims 1 to 8 to a patient in need thereof.

17. The invention as hereinbefore described.
